(19) United States
(12) Patent Application Publication
(10) Pub. No.: US 2010/0035873 A1
(43) Pub. Date: Feb. 11, 2010

(54) Diaryl Compounds and Uses Thereof

(75) Inventors: Patrick Verhoest, Old Lyme, CT (US); Matthew Merrill Hayward, Old Lyme, CT (US); Vinod Parikh, Mystic, CT (US); Stanton Furst McHardy, Helotes, TX (US); Stafford McLean, Stonington, CT (US)

Correspondence Address: PFIZER INC.;PATENT DEPARTMENT, Bld 114 M/S 114, EASTERN POINT ROAD, GROTON, CT, 06340, US

(73) Assignee: Pfizer Inc

(21) Appl. No.: 12/489,902

(22) Filed: Jun. 23, 2009

Related U.S. Application Data
(60) Provisional application No. 61/075,420, filed on Jun. 25, 2008.

(51) Int. Cl. A61K 31/535; C07D 401/02; A61K 31/4439; C07D 265/30

(52) U.S. Cl. 514/231.2; 546/276.4; 514/343; 544/106

(57) ABSTRACT

The invention relates to derivatives of a compound of formula I:

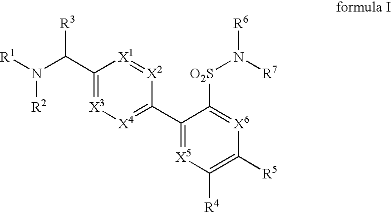

wherein R¹ to R⁷ and X¹ to X⁶ are as defined herein.

The invention relates to the uses thereof for treating diseases, conditions and/or disorders mediated by kappa opioid receptors (KORs). Specifically, the compounds are selective antagonists of KORs and are highly selective to KORs relative to mu and delta opioid receptors.

### FIELD OF THE INVENTION

The present invention relates to diaryl derivatives that are substituted phenyl-phenyl, phenyl-heteroaryl, or heteroaryl-heteroaryl compounds and the uses thereof for treating diseases, conditions and/or disorders mediated by kappa opioid receptors (KORs). Specifically, the compounds are selective antagonists of KORs and are selective to KORs relative to mu and delta (μ and δ) opioid receptors.

### BACKGROUND

The compounds of the invention can be used for the treatment of CNS diseases, conditions, and/or disorders described herein alone or in combination with any other pharmaceutical agent, where the compound of formula I and/or the agent may be a pharmaceutically acceptable salt thereof. The other agent includes:

an antimanic agent (mood stabilizers) (including Lithium, Carbamazepine {5H-dibenz[b,f]azepine-5-carboxamide}, Depakote {divalproex sodium dissociates to the valproate ion, chemically known as sodium hydrogen bis(2-propylpentanoate)}, and Lamotrigine {3,5-diamino-6-(2,3-dichlorophenyl)-as-triazine}, and Abilify (also known as aripiprazole that is an atypical antipsychotic),

an atypical antipsychotic (including ziprasidone {5-[2[-4-(1,2-benzisothiazol-3-yl)piperazin-1-yl])ethyl-]-6-chloro-1,3-dihydro-2H-indol-2-one (see, e.g., U.S. Pat. Nos. 4,831,031, 5,312,295, 6,387,904, 6,245,765, and 6,245,766 and European Patent application EP901781, published Mar. 17, 1999)}; olanzapine {2-methyl-4-(4-methyl-1-piperazinyl)-10H-thieno-[2,3-b][1,5]benzodiazepine (see, e.g., U.S. Pat. No. 5,229,382)}; clozapine {(8-chloro-11-(4-methyl-1-piperazinyl)-5H-dibenzo[b,e][1,4]diazepine (see, e.g., U.S. Pat. No. 3,539,573; see also Hanes et al., Psychopharmacol Bull. 24, 62 (1998))}; risperidone {3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one (see, e.g., U.S. Pat. No. 4,804,663)}; sertindole {1-[2-[4-[5-chloro-1-(4-fluorophenyl)-1H-indol-3-yl]-1-piperidinyl]ethyl]limidazolidin-2-one (see, e.g., U.S. Pat. Nos. 4,710,500; 5,112,838 and 5,238,945)}; quetiapine {(2-[2-(4-dibenzo[b,f][1,4]thiazepin-11-yl-1-piperazinyl)ethoxy]ethanol, see e.g., U.S. Pat. No. 4,879,288)}; aripiprazole {(7-{4-[4-(2,3-dichlorophenyl)-1-piperazinyl-butoxy}-3-4-dihydrocarbostyril or 7-{4-[4-(2,3-dichlorophenyl)-1 piperazinyl]-butoxy}-3,4-dihydro-2(1H)-quinolinone (see e.g., U.S. Pat. Nos. 4,734,416 and 5,006,528)]; amisulpride {(4-amino-N-[1-ethyl-2-pyrrolidinyl)methyl]-5-(ethylsulfonyl)-2-methoxybenzamide (see, e.g., U.S. Pat. No. 4,401,822; see also P. Protais, et al. Neuropharmacol, 24, 861 (1985))}; mirtazepine {1,2,3,4,10,14b-hexa-hydro-2-methylpyrazino[2,1-a]pyrido[2,3-c]-[2]benzazepine (see, U.S. Pat. No. 4,062,848)}; and asenapine {trans-5-chloro-2-methyl-2,3,3a,12b-tetrahydro-1H-dibenz-[2,3:6,7]oxepino[4,5-c]pyrrole (see, e.g., U.S. Pat. Nos. 4,145,434 and 5,763,476))}, or

a 5-HT re-uptake inhibitor (including sertraline, paroxetine, fluoxetine, citalopram, and escitalopram).

U.S. Pat. No. 6,974,824 discusses kappa opioid receptor antagonists that are said to yield significant improvements in functional binding assays to kappa opioid receptors relative to nor-BNI, and the use of these antagonists in treatment of disease states that are ameliorated by binding of the kappa opioid receptor such as heroin or cocaine addictions.

U.S. Pat. No. 6,559,159 discusses kappa opioid receptor antagonists and the use of these antagonists in treatment of disease states that are said to ameliorated by binding of the kappa opioid receptor such as heroin or cocaine addictions.

U.S. Pat. No. 6,548,637 discusses compounds, and pharmaceutical preparations thereof, that bind selectively to mammalian opioid receptors where the present set of compounds are said to comprise full agonists, partial agonists, and antagonists of mammalian opioid receptors.

U.S. Pat. No. 6,528,518 discusses the treatment of depression using kappa opioid receptor antagonists.

U.S. Pat. No. 5,780,479 discusses a method for treating an individual afflicted with an impulse-control disorder by administering thereto an amount of one or more opioid receptor antagonists.

U.S. Pat. No. 5,727,570 discusses a method of treatment of humans suffering from hyperlipidemia which comprises administering, by a pharmaceutically effective mode, a drug composition selected from the group consisting of opiate antagonists, and drugs which substantially equally reduce the amounts of catecholamines bound to all catecholamine binding sites.

U.S. Pat. No. 5,585,348 discusses a method of preventing hyperalgesia and other undesirable side-effects associated with the administration of growth factor, including nerve growth factor, utilizing an antagonist capable of inactivating excitatory opioid receptor-mediated functions on neurons in the nociceptive pathway. In addition, the invention relates to a composition comprising a growth factor and an antagonist capable of inactivating excitatory opioid receptor-mediated functions on neurons in the nociceptive pathway.

U.S. Pat. No. 5,141,962 discusses compounds claimed to have dissociated antagonist affinity for kappa opiate receptor.

U.S. Pat. No. 5,025,018 discusses methods of inducing opiate-receptor antagonistic activity in a patient suffering from ischemic or traumatic central nervous system injury by administering to said patient an effective amount of an opiate-receptor antagonist having enhanced activity at the kappa-opiate receptor suitable to permit the induction of opiate-receptor antagonistic activity.

U.S. Pat. No. 4,906,637 discusses methods of inducing opiate-receptor antagonistic activity in a patient suffering from ischemic or traumatic brain injury by administering to said patient an effective amount of an opiate-receptor antagonist having enhanced activity at the kappa-opiate receptor suitable to permit the induction of opiate-receptor antagonistic activity.

WO 2007/100335 discusses methods of treating mood disorders, such as manic disorders, and stabilizing moods by administering a kappa agonist or partial agonist to a subject in need thereof.

The mesolimbic dopamine system, which originates in the ventral tegmental area and projects to the nucleus accumbens (NAc), is involved in the pleasurable (hedonic) and rewarding effects of a variety of substrates, including drugs of abuse, food, and sexual behavior. Drugs of abuse cause complex neuroadaptations in this system, some of which are associated with altered drug sensitivity. One neuroadaptation involves cAMP response element-binding protein (CREB), a transcription factor that is activated in striatal regions by psychostimulants. CREB in the NAc appears to regulate the rewarding and aversive effects of cocaine. Stimulation of cAMP-dependent protein kinase A (PKA), which activates CREB, in the NAc decreases cocaine reward. Similarly, elevation of CREB expression in the NAc decreases cocaine reward and makes low doses of the drug aversive. Conversely, blockade of PKA activity or overexpression of a dominant-negative CREB, which functions as a CREB antagonist, in the NAc increase cocaine reward. These findings suggest that CREB activation in the NAc counteracts drug reward and increases drug aversion.

Cocaine alters neuronal excitability and neurotransmitter levels in the brain, particularly the mesolimbic dopamine system. Cocaine withdrawal is accompanied by signs of depression and other mood disorders in humans. The biological basis of mood disorders like depression is not understood, but may be caused by genetic and environmental factors. Physically and emotionally stressful events can also influence the etiology of depression, possibly causing subtle brain changes and alterations in gene expression. Thus, depression may have an important acquired component, caused by neuroadaptations in response to environment and experience.

The therapeutic actions of antidepressants appear to involve neuroadaptations. Most antidepressant treatments (including tricyclic and atypical antidepressants, selective serotonin reuptake inhibitors, electroconvulsive therapy) have common actions on components of the cAMP pathway. Common actions include activation of PKA and the transcription factor CREB in the hippocampus, a brain region associated with emotion. CREB plays a critical role in the expression of numerous genes. Understanding causal relations among CREB function, gene expression, and the therapeutic effects of antidepressants might provide explanations for why antidepressants require sustained treatment for effectiveness. Additionally, because some genes regulated by CREB may be therapeutic while others may be pathophysiological, a more general understanding of the role CREB in behavior might help to elucidate the biological basis of depressive syndromes.

Many of the researchers studying depression are focused on the hippocampus. Many antidepressants increase the level of CREB in the hippocampus. In this region, it is believed that increasing CREB activity is beneficial, because CREB controls some growth factors (e.g., BDNF) in the brain. See D'Sa C, Duman R S., Bipolar Disord. 2002: 4: 183-94, providing a discussion between CREB and antidepressant activity.

Many of the researchers studying depression are focused on the hippocampus. Many antidepressants increase the level of CREB in the hippocampus. In this region, it is believed that increasing CREB activity is beneficial, because CREB controls some growth factors (e.g., BDNF) in the brain. However, there is no evidence that increasing CREB in the hippocampus is associated with the therapeutic effects of antidepressants.

Although much research on the molecular mechanisms of depression and antidepressant actions has focused on the hippocampus, the NAc may also have relevance. This basal forebrain region is innervated by dopamine neurons of the ventral tegmental area, as well as by noradrenergic and serotonergic inputs. The NAc contributes importantly to the pleasurable effects of food, sexual behavior, novelty, and addictive drugs.

Most current antidepressants act primarily on brain levels of noradrenaline or serotonin. There is some evidence that dopamine systems might be involved in depressive syndromes. Blocking dopamine receptors in the brain causes anhedonia (a decreased ability to experience pleasure), a defining feature of depression. Nomifensine, a dopamine reuptake inhibitor, was a clinically effective antidepressant, further implicating dopaminergic dysfunction in depression. Nomifensine was taken off the market because it caused lethal allergic reactions in some people.

Buprenorphine (BUP), a partial mu agonist/weak partial kappa agonist, was reported to be effective in the pharmacological treatment of affective disorders. A double blind investigation showed BUP to induce strong antidepressant effects in patients with endogenous depression (Emrich, et al., Ann. NY Acad. Sci., 1982, v398, p 108). Additionally, depressive symptoms were found to be significantly decreased with BUP treatment in heroin addicted patients who were depressed at intake (Kosten, J, Subst. Abuse Treat, 1990, v1, p 51).

Recently, Gerra et al. (Gerra et al, Prog. Neuropsychopharmacol Biol. Psychiatry, 2006, v30, p 265) reported a better outcome in heroin addicts co-morbid for depression, as a result of the improvement of depressive symptoms, that was tentatively attributed to the specific pharmacological profile of BUP. In contrast to antagonists, kappa receptor agonists, such as butorphanol and enadoline have been reported to increase dysphoria, confusion, sedation and to produce feelings of depersonalisation in humans (Greenwald and Stitzer, Drug Alcohol Depend., 1998, v1, p 17; and Walsh et al., Psychopharmacology 2001, v157, p 151), thus supporting the use of antagonists or partial agonists in depressed subjects.

Preclinically, immobilization stress, forced swim, or induction of learned helplessness (LH) increase dynorphin (endogenous ligand for kappa receptors) immunoreactivity in specific subregions of the hippocampus, as well as NAc. Conversely, KOR antagonists to these regions produces an antidepressant response in the LH model of depression (Shirayama et al., J. Neurochem., 2004, v90, p 1258), consistent with antidepressant effects observed after systemic administration of kappa receptor antagonists.

Changes in dynorphin levels in the NAc in response to stress may also be noteworthy. Most depressed patients exhibit a reduced ability to experience pleasure (anhedonia) and loss of motivation. Reward is mediated by the dopaminergic neurons located in the ventral tegmental area (VTA) that project to the NAc and is modulated (inhibited) by the kappa receptors located directly on dopamine cells. Thus, blockade of kappa receptors in the NAc has antidepressant activity in several animal models and likely to blunt the decreased reward associated with excessive stimulation of kappa receptors. Dynorphin upregulation in the NAc shell has been shown to be stimulated by stress and various drugs of abuse, and to cause anhedonia-like effects (Newton et al. J. Neurosci., 2002, v22, p 10833).

Depressed patients perform significantly worse than controls in learning and memory tasks (Hasler et al., Neuropsychopharm., 2004 v 29 p 1765 and Zakzanis, Neuropsychiatry Neuropsychol. Behav. Neurol., 1998 v11, p 111) consistent with imaging studies suggesting decreased hippocampal volume. Hippocampal changes are also observed in bipolar disorder (Frey, et al., Beh. Pharmacol. Vol. 18(5-6), pp 419-430) with some evidence of reduced glutamatergic transmission that may contribute to impairments in learning and memory observed in bipolar patients. Interestingly, dynorphin is co-localized with glutamate in granule cells of the hippocampus and exerts a potent inhibitory control over glutamate release in the hippocampus. Furthermore, a kappa receptor antagonist can potentiate LTP induced by prolonged stimulation (Terman et al, J. Neurosci., 2000, v20, p 4379) suggesting that a kappa antagonist may facilitate learning and memory.

Although many opioid receptor antagonists are known, there remains a need to identify compounds having improved selectivity at the KOR over other receptors.

### SUMMARY

Compounds of Formula (I) have been found to act as selective antagonists at the KOR, and, therefore, may be used in the treatment of diseases, conditions and/or disorders that benefit from such antagonism (e.g., diseases/disorders/conditions related to obesity and obesity-related co-morbidities in addition to those related to the central nervous system). In particular, the compounds of Formula (I) provide selectivity at the KOR.

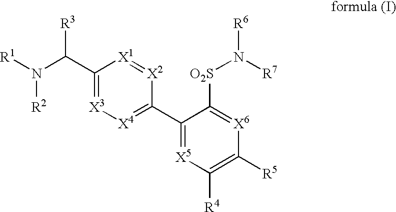

wherein the variables are described herein.

In an embodiment of the present invention, a method for treating, or preparing a medicament to treat, a disease, condition and/or disorder that is mediated by selectively antagonizing the KOR over the mu and delta opioid receptors in animals that include the step of administering to an animal (preferably, a human) in need of such treatment a therapeutically effective amount of a compound of the present invention (or a pharmaceutical composition thereof) to treat any disease, condition, or disorder mediated by antagonizing the kappa opioid receptor.

Diseases, conditions, and/or disorders mediated by selectively antagonizing the kappa opioid receptor include any one individually or combination of any of the following: schizophrenia including negative symptoms; schizophreniform disorder; schizoaffective disorder including of the delusional type or the depressive type; delusional disorder; substance-induced psychotic disorder; personality disorder of the paranoid type; personality disorder of the schizoid type; panic disorder; phobias; obsessive-compulsive disorder; stress disorders; generalized anxiety disorder; movement disorders involving Huntington's disease; dyskinesia associated with dopamine agonist therapy; Parkinson's disease; restless leg syndrome; disorders comprising as a symptom thereof a deficiency in cognition; dementias; mood disorders and episodes in a mammal; anxiety or psychotic disorders including schizophrenia, of the paranoid, disorganized, catatonic, undifferentiated, or residual type; delusional disorder; personality disorder of the paranoid type, of the schizoid type, or agoraphobia; post-traumatic stress disorder; acute stress disorder; chemical dependencies including alcohol, amphetamine, cocaine, heroin, phenobarbital, opiate, nicotine and benzodiazepines addiction; deficiency in memory, intellect, or learning and logic ability; reduction in any particular individual's functioning in one or more cognitive aspects; age-related cognitive decline; dementia; Alzheimer's disease; multi-infarct dementia; alcoholic dementia or other drug-related dementia; dementia associated with intracranial tumors or cerebral trauma; dementia associated with Huntington's disease or Parkinson's disease; AIDS-related dementia; delirium; amnestic disorder; mental retardation; a learning disorder including reading disorder, mathematics disorder, or a disorder of written expression; attention-deficit/hyperactivity disorder; mood disorders or mood episodes; a manic or mixed mood episode; a hypomanic mood episode; a depressive episode with atypical features; a depressive episode with melancholic features; a depressive episode with catatonic features; a mood episode with postpartum onset; post-stroke depression; dysthymic disorder; minor depressive disorder; premenstrual dysphoric disorder; post-psychotic depressive disorder of schizophrenia; a major depressive disorder superimposed on a psychotic disorder; delusional disorder or schizophrenia; a bipolar disorder including bipolar I disorder, bipolar II disorder, cyclothymic disorder, hypertension, and depression; depression in cancer patients, Parkinson's patients, infertile women, and pediatrics; depression as a single episode depression or recurrent episodes; including depression associated with postmyocardial infarction, subsyndromal symptomatic depression, induced by child abuse, post partum depression, and major depression of the mild, moderate or severe type; avoidant personality disorder; premature ejaculation; eating disorders including anorexia nervosa and bulimia nervosa; obesity; cluster headache; migraine; pain; neuroleptic-induced parkinsonism and tardive dyskinesias; endocrine disorders; hyperprolactinaemia; vasospasm; vasospasm in the cerebral vasculature; cerebellar ataxia; gastrointestinal tract disorders involving changes in motility and secretion; mania; premenstrual syndrome; fibromyalgia syndrome; stress incontinence; Tourette's syndrome; trichotillomania; kleptomania; male impotence; cancer; small cell lung carcinoma; chronic paroxysmal hemicrania; and headache associated with vascular disorders. Of most particular interest is schizophrenia, using the compounds of formula in combination with an atypical antipsychotic and also depression and/or bipolar using the compounds of formula I as monotherapy or in combination with an antimanic agent (mood stabilizers, including Lithium, Carbamazepine, Valproate, Lamotrigine, and Abilify), or with a 5-HT re-uptake inhibitor (including sertraline).

The phrase “therapeutically effective amount” means an amount of a compound of the present invention that (i) treats or prevents the particular disease, condition, or disorder; (ii) attenuates, ameliorates, or eliminates one or more symptoms of the particular disease, condition, or disorder; or (iii) prevents or delays the onset of one or more symptoms of the particular disease, condition, or disorder described herein.

The term “animal” refers to humans (male or female), companion animals (e.g., dogs, cats and horses), food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both preventative, i.e., prophylactic, and palliative treatment.

The terms “modulating opioid receptor activity” or “opioid-mediated” refers to the activation or deactivation of the mu, kappa and/or delta opioid receptors.

The term “compounds of the present invention” refer, unless specifically identified otherwise, to compounds of formula (I) and pharmaceutically acceptable salts of the compounds, and hydrates of the compounds thereof and salts thereof, as well as, all stereoisomers (including diastereomers and enantiomers), tautomers and isotopically labeled compounds.

### DETAILED DESCRIPTION

One aspect of the present invention is a compound of formula I:

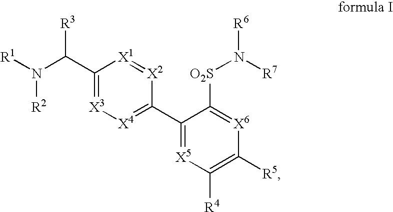

or a pharmaceutically acceptable salt thereof, wherein

R¹ is C₁₋₆alkyl, C₂₋₄alkyl-O—C₁₋₂alkyl, a 7-, 8-, or 9-membered bridged bicyclic carbocyclic ring, a fused bicyclic carbocyclic ring, —(CH₂)ₐ-phenyl, —(CH₂)ₐ-heteroaryl, or —(CH₂)ₐheterocycloalkyl, wherein the bridged ring, the fused ring, phenyl, heteroaryl, or heterocycloalkyl is unsubstituted or substituted with 1, 2, or 3 substituents independently selected from halogen, OH, C₁₋₃alkyl, O—C₁₋₃alkyl, NH₂, NHC₁₋₃alkyl, or N(C₁₋₃alkyl)₂;

R² is H or C₁₋₄alkyl;

or

R¹ and R² are taken together with the nitrogen to which they are attached to form a mono- or bicyclic N-ring, where said N-ring is a 4- to 7-membered mono-cyclic heterocycloalkyl ring; a fused bicyclic heterocyclic ring; or a 7-, 8-, or 9-membered bridged bicyclic heterocyclic ring, wherein said N-ring is unsubstituted or substituted with 1, 2, or 3 substituents independently selected from halogen, OH, —CN, C₁₋₃alkyl, C₁₋₃alkyl-OH, O—C₁₋₃alkyl, C₁₋₃alkyl-O—C₁₋₃alkyl, NH₂, NHC₁₋₃alkyl, or N(C₁₋₃alkyl)₂;

a is 0, 1, or 2;

R³ is H, or C₁₋₃alkyl; or

when X¹ or X³ is >(C(R⁸))—, R³ may be taken together with R⁸ of one of X¹ or X³ and the carbon atoms to which they are attached to form a 5- or 6-membered saturated, or partially saturated ring, wherein one carbon atom in said 5- or 6-membered ring may be a heteroatom selected from —O—, —N(H)—, —N(C₁₋₃alkyl)-, and >N—, and wherein said ring is unsubstituted or substituted where valency permits with 1 substituent selected from halogen, —CN, or —C₁₋₃alkyl;

R⁴ is H, halogen, CN, C₁₋₃alkyl, or OC₁₋₃alkyl;

R⁵ is H, halogen, CN, C₁₋₃alkyl, or OC₁₋₃alkyl;

R⁶ is C₁₋₄alkyl, or C₂₋₄alkyl-O—C₁₋₂alkyl;

R⁷ is H, or C₁₋₄alkyl;

or

R⁶ and R⁷ are taken together with the nitrogen to which they are attached to form a mono- or bicyclic SN-ring, where said SN-ring is a 4- to 7-membered mono-cyclic heterocyclic ring containing 0 or 1 additional heteroatom selected from O, NH, or NC₁₋₃alkyl, or a 7-, 8-, or 9-membered bridged bicyclic heterocyclic ring, wherein said SN-ring is unsubstituted or substituted with 1 or 2 substituents independently selected from halogen, OH, —CN, C₁₋₃alkyl, C₁₋₃alkyl-OH, O—C₁₋₃alkyl, C₁₋₃ alkyl-O—C₁₋₃ alkyl, NH₂, NHC₁₋₃alkyl, or N(C₁₋₃alkyl)₂;

X¹, X², X³, X⁴, X⁵, and X⁶ are independently >(C(R⁸))— or >N—; and

each R⁸ is independently H, halogen, —CN, —C₁₋₃alkyl, —OC₁₋₃alkyl, provided that when X¹ or X³ is >(C(R⁸))—, one R⁸ of X₁ or X₃ may be taken together with R³ and the carbon atoms to which they are attached to form said 5- or 6-membered saturated, or partially saturated ring.

In the invention, it is preferable that X¹, X², X³, and X⁴ are >(C(R⁸))— wherein R⁸ is selected from any definition discussed herein, and also include wherein each R⁸ is independently H, halogen, —CN, —C₁₋₃alkyl, or —OC₁₋₃alkyl.

Another aspect of the invention is where R⁶ and R⁷ are taken together with the nitrogen to which they are attached to form the monocyclic SN-ring wherein said SN-ring is unsubstituted or substituted with C₁₋₃alkyl. Preferred monocyclic moieties making this SN-ring include pyrrolidinyl or morpholinyl wherein said SN-ring is unsubstituted or substituted with methyl.

Another aspect of the invention is wherein R¹ is C₁₋₆alkyl or the 7-membered bridged bicyclic carbocyclic ring, and R² is H.

Yet, another aspect of the invention is wherein R¹ and R² are taken together with the nitrogen to which they are attached to form the 5- to 6-membered mono-heterocycloalkyl ring, or the 8-membered bridged bicyclic heterocyclic ring, wherein said N-ring is unsubstituted or substituted with 1 or 2 substitutents independently selected from OH, methyl, or methoxy.

Another aspect of the invention is wherein X⁵ is <N— and X¹, X², X³, X⁴, and X⁶ are >(C(R⁸))—, or, alternatively X⁶ is >N— and X¹, X², X³, X⁴, and X⁵ are >(C(R⁸))—, wherein R⁸ is independently H, halogen, —CN, —C₁₋₃alkyl, or —OC₁₋₃alkyl.

Another aspect of the invention is wherein X¹ or X³ are both >(C(R⁸))— and only one R⁸ of X¹ and X³ is H and the other R⁸ is halogen, —CN, —C₁₋₃alkyl, or —OC₁₋₃alkyl. Another aspect of the invention is wherein at least one of X¹ or X³ is >(C(R⁸))— and R³ of formula I forms a ring with R⁸ of X¹ or X³ and the carbon atoms to which they are attached to form a 5- or 6-membered saturated, or partially saturated ring, wherein one carbon atom in said 5- or 6-membered ring may be a heteroatom selected from —O—, —N(H)—, —N(C₁₋₃alkyl)-, and >N—, and wherein said ring is unsubstituted or substituted where valency permits with 1 substituent selected from halogen, —CN, or —C₁₋₃alkyl.

The invention includes the free base or a pharmaceutically acceptable salt of every example of compounds of formula I, by itself or in a group of any combination of the compounds of formula I prepared herein.

“Halogen” and “halo” and the like include fluoro, chloro, bromo and iodo.

The term “alkyl”, as used herein, unless indicated otherwise, includes straight or branched alkyl groups of C_{e-g} number of carbons where e is the least number of carbon atoms and g is the largest number of carbon atoms possible. When an alkyl moiety contains at least 3 carbon atoms, said alkyl includes cycloalkyl moieties. Each alkyl moiety may be substituted with up to three substituents independently selected from halogen, OH, —CN, O—C₁₋₃alkyl, NH₂, NHC₁₋₃alkyl, or N(C₁₋₃alkyl)₂, wherein the alkyl moieties that are substituents are not further substituted unless otherwise indicated. Therefore, non-limiting examples of C₁₋₄alkyl include methyl, trifluoromethyl, ethyl, n-propyl, i-propyl, cyclopropyl, methyl-cyclopropyl, t-butyl, and cyclobutyl.

The term “heterocycloalkyl” as used herein, unless indicated otherwise, includes a 4- to 7-membered saturated cycloalkyl moiety in which up to two carbon atoms are replaced with a nitrogen, O, or S atom or any combination thereof. When nitrogen replaces an atom, to satisfy velancy requirements, the nitrogen is substituted with H or C₁₋₃alkyl or is a point of attachment when heterocycloalkyl is a substituent or is created by two substituents combining to form a heterocycloalkyl moiety, e.g., when R¹ and R² are taken together with the atom to which they are attached to make pyrrolidinyl. Non-limiting examples of heterocycloalkyl include oxetanyl, tetrahydrofuranyl, tetrahydropyran, azetidinyl, pyrrolidinyl, pyrazolidinyl, piperidinyl, dioxanyl, morpholinyl, thiomorpholinyl, and piperazinyl.

The term “aryl” refers to carbocyclic aromatic moieties having a single ring (e.g., phenyl).

The term “heteroaryl” as used herein, unless indicated otherwise, is a 5- to 6-membered ring that is aromatic or partially saturated, where at least one heteroatom is present and no more that three heteroatoms are present, wherein the heteroatom is nitrogen, O, or S or any combination thereof. When nitrogen replaces an atom, to satisfy velancy requirements, the nitrogen is substituted with H or C₁₋₃ alkyl or is the point of attachment when heteroaryl is a substituent or is formed by two substituents combining to form a heteroaryl moiety. Non-limiting examples of heteroaryl include pyridyl, pyrimidyl, pyridazyl, pyrazinyl, furanyl, thiophenyl, pyrrolyl, pyrrolinyl, oxazolyl, isoxazolyl, thiazolyl, isothiazolyl, imidazolyl, imidazolinyl, pyrazolyl, pyrazolinyl, pyranyl, and azapinyl.

A fused bicyclic carbocyclic ring as used herein, unless indicated otherwise, has the two rings that share adjacent carbon atoms. The fused rings form a 5-6 or 6-6 fused bicyclic ring. In the fused ring, unless otherwise specified, either ring may be aromatic, or partially or fully saturated. A non-limiting example of a fused aromatic ring moiety is naphthyl. A non-limiting example of a saturated or partially saturated fused ring moiety is decahydronaphthyl and tetrahydronaphthyl, respectively. Other non-limiting examples include indanyl.

A fused bicyclic heterocyclic ring as used herein, unless indicated otherwise, is a bicyclic fused ring where up to 3 atoms are independently selected from 0, nitrogen, or S or any combination thereof in either ring, including the atom where the rings are fused where valency permits. When nitrogen is the heteroatom, to satisfy valency requirements, it is substituted with H or C₁₋₄alkyl, or is a point of attachment when a fused bicyclic heterocyclic ring is a substituent or is the point of attachment when formed by two substituents combining to form a fused bicyclic heterocyclic moiety.

A bridged bicyclic carbocyclic ring as used herein, unless indicated otherwise, is formed when two rings do not share adjacent carbon atoms to make the bicyclic ring. The bridged ring contains from 7 to 9 carbon atoms. Non-limiting examples include bicyclo[2.2.1]heptanyl and bicyclo[3.1.1]heptanyl.

A bridged bicyclic heterocyclic ring as used herein, unless indicated otherwise, is a bridged bicyclic carbocyclic ring where 1 to 2 carbon atoms are replaced by a nitrogen atom that is substituted where valency requires by H or C₁₋₃alkyl or provides the point of attachment for the ring when the ring is a substituent or is formed, for example, when R¹ and R² or R⁶ and R⁷ combine to form a bridged bicyclic heterocyclic ring. Non-limiting examples include quinuclidinyl, 6-aza-[3.2.1]-octanyl, 2-azabicyclo[2.2.1]heptanyl, 3-azabicyclo[3.3.2]decanyl, 2-azabicyclo[2.2.2]octanyl, and 3-azabicyclo[3.2.1]octanyl.

Another aspect of the present invention is a pharmaceutical composition that comprises (1) a compound of the present invention, and (2) a pharmaceutically acceptable excipient, diluent, or carrier. Preferably, the composition comprises a therapeutically effective amount of a compound of the present invention. The composition may also contain at least one additional pharmaceutical agent (described herein).

In yet another embodiment of the present invention, a method for treating, or preparing a medicament to treat, a disease, condition and/or disorder that is mediated by selectively antagonizing the KOR over the mu and delta opioid receptors in animals that include the step of administering to an animal (preferably, a human) in need of such treatment a therapeutically effective amount of a compound of the present invention (or a pharmaceutical composition thereof) to treat any disease, condition, or disorder mediated by antagonizing the kappa opioid receptor.

Diseases, conditions, and/or disorders mediated by selectively antagonizing the kappa opioid receptor include any one individually or combination of any of the diseases discussed herein.

Compounds of the present invention may be administered in combination with other pharmaceutical agents. The combination therapy may be administered as (a) a single pharmaceutical composition which comprises a compound of the present invention, at least one additional pharmaceutical agent described herein and a pharmaceutically acceptable excipient, diluent, or carrier; or (b) two separate pharmaceutical compositions comprising (i) a first composition comprising a compound of the present invention and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions may be administered simultaneously or sequentially and in any order.

Compounds of the present invention may be synthesized by synthetic routes that include processes analogous to those well-known in the chemical arts, particularly in light of the description contained herein. The starting materials are generally commercially available or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

The following schemes outline the general procedures one could use to provide compounds of the present invention.

The compounds of this invention can be prepared starting from the aryl sulphonyl chloride with a group ortho that can be utilized to couple in a palladium-catalyzed reaction such as a Suzuki coupling (see for example Miyaura, N.; et al Chem. Rev. 1995, 95, 2457). Standard ortho groups for R¹⁰ are a chloride, bromide, iodide or boronic acid. These compounds are commercially available or can be prepared by one who is skilled in the art.

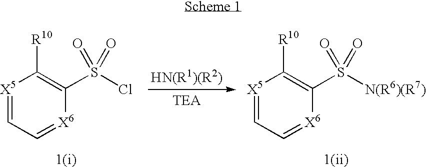

Treatment of the sulphonyl chloride with an organic tertiary amine base in organic solvent such as tetrahydrofuran followed by the addition of the secondary or primary amine readily affords the sulfonamide.

When X⁶ is >N— and R¹⁰ is H, the halogenated intermediate 2(ii) can be prepared as outlined in Scheme 2:

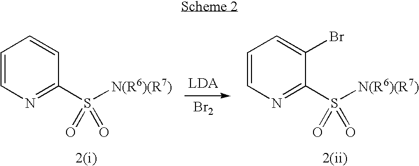

The sulfonamide can be treated with LDA in tetrahydrofuran or potentially an alternative organic aprotic solvent and the anion quenched with bromine to provide the Suzuki coupling partner 2(ii). If either R⁶ or R⁷ are a proton, an additional equivalent of base (LDA) will be required to carry out this sequence.

Suzuki couplings are well precendented in the literature (see for example Miyaura, N.; et al Chem. Rev. 1995, 95, 2457) and are very effective in coupling aryl halides with aryl boronic acids. This can be accomplished with a variety of palladium catalysts and ligands which can improve the coupling or aryl chlorides (Buchwald et al., Angwandte Chemie, International addition, 1999, 38(16) 2413-2416). One of these ligands and palladium catalysts is DPPF ([1,1′-Bis(diphenylphosphino)ferrocene]dichloropalladium (II).

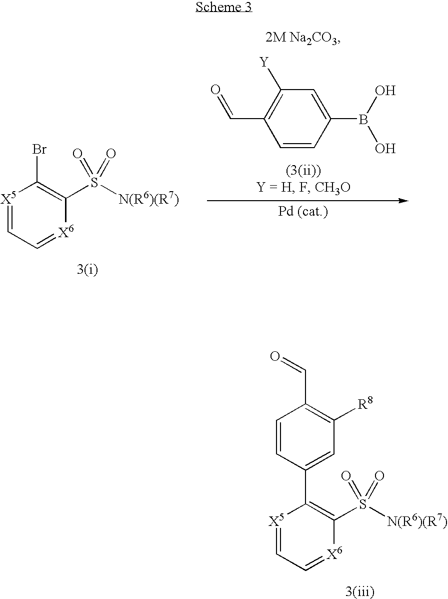

Compounds like 3(i) (Scheme 3) can be coupled to compounds like 3(ii) utilizing DPPF as the catalyst/ligand source. This can be conducted in a variety of organic solvents with one such being dimethylacetamide or ethylene glocol. These reactions can be conducted at ambient temperature or may require heating, either condition readily being determined by one of ordinary skill in the art.

Compounds like 3(iii) can be converted to compounds of Formula I through a standard reductive amination (Scheme 4). Treatment of aldehyde 3(iii) with a secondary or primary amine in an organic solvent such as tetrahyrdofuran in the presence of catalytic acid (e.g., acetic acid) followed by addition of a reducing reagent affords the desired compounds of formula I. The variety of reducing agents can be utilized, one such reagent is sodium triacetoxy borohydride.

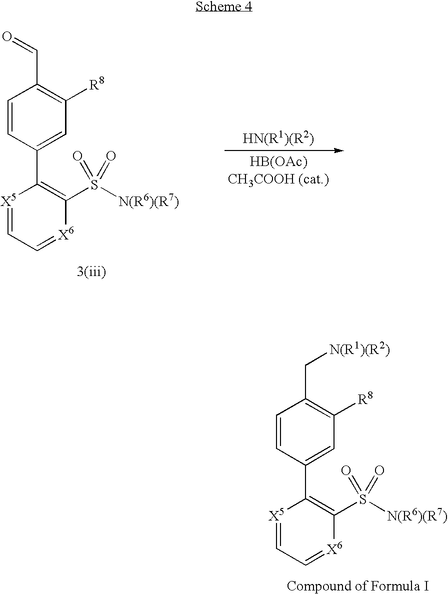

When one of X¹-X⁴ is N, the pyridyl boronic acid aldehyde is commercially available and coupled as described in Scheme 4.

The compounds of the present invention may be isolated and used per se or in the form of any pharmaceutically acceptable salt. The term “salts” refers to inorganic and organic salts of a compound of the present invention. These salts can be prepared in situ during the final isolation and purification of a compound, or by separately reacting the compound with a suitable organic or inorganic acid or base and isolating the salt thus formed. Representative salts include the hydrobromide, hydrochloride, hydroiodide, sulfate, bisulfate, nitrate, acetate, trifluoroacetate, oxalate, besylate, palmitate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, e.g., Berge, et al., J. Pharm. Sci., 66, 1-19 (1977).

The compounds of the present invention may contain asymmetric or chiral centers, and, therefore, exist in different stereoisomeric forms. It is intended that all stereoisomeric forms of the compounds of the present invention as well as mixtures thereof, including racemic mixtures, form part of the present invention. In addition, the present invention embraces all geometric and positional isomers. For example, if a compound of the present invention incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the present invention may be atropisomers (e.g., substituted biaryls) and are considered as part of this invention. Enantiomers can also be separated by use of a chiral HPLC column.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

The present invention also embraces isotopically-labeled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

Compounds of the present invention are useful for treating diseases, conditions and/or disorders modulated by the mu, kappa and/or delta opioid receptors; therefore, another embodiment of the present invention is a pharmaceutical composition comprising a therapeutically effective amount of a compound of the present invention and a pharmaceutically acceptable excipient, diluent or carrier. The compounds of the present invention (including the compositions and processes used therein) may also be used in the manufacture of a medicament for the therapeutic applications described herein.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The present invention further provides a method of treating diseases, conditions and/or disorders modulated by the opioid receptor(s) in an animal that includes administering to an animal in need of such treatment a therapeutically effective amount of a compound of the present invention or a pharmaceutical composition comprising an effective amount of a compound of the present invention and a pharmaceutically acceptable excipient, diluent, or carrier. The method is particularly useful for treating diseases, conditions and/or disorders that benefit from antagonizing the mu, kappa and/or delta opioid receptors.

For a normal adult human having a body weight of about 100 kg, a dosage in the range of from about 0.001 mg to about 10 mg per kilogram body weight is typically sufficient, preferably from about 0.005 mg/kg to about 5.0 mg/kg, more preferably from about 0.01 mg/kg to about 3 mg/kg. However, some variability in the general dosage range may be required depending upon the age and weight of the subject being treated, the intended route of administration, the particular compound being administered and the like. The determination of dosage ranges and optimal dosages for a particular patient is well within the ability of one of ordinary skill in the art having the benefit of the instant disclosure. It is also noted that the compounds of the present invention can be used in sustained release, controlled release, and delayed release formulations, which forms are also well known to one of ordinary skill in the art.

The compounds of this invention may also be used in conjunction with other pharmaceutical agents for the treatment of the diseases, conditions and/or disorders described herein. Therefore, methods of treatment that include administering compounds of the present invention in combination with other pharmaceutical agents are also provided. Suitable pharmaceutical agents that may be used in combination with the compounds of the present invention include anti-obesity agents.

Another aspect of the present invention is the treatment of central nervous system diseases, disorders, and/or conditions as discussed herein.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England).

### Pharmacological Testing

The practice of the instant invention for treating the diseases or conditions discussed herein can be evidenced by activity in at least one of the protocols described hereinbelow.

In Vitro Biological Assays

Binding Assay

Binding assays on membranes from CHO cells expressing either human kappa, mu or delta opioid receptors were performed according to standard procedures. Briefly, frozen cell paste (70-80-mgs per 96 well plate) is homogenized in 50 mM Tris HCl buffer (pH 7.4 @ 4 degrees C.) containing 2.0 mM MgCl2 using a Polytron and spun in a centrifuge at 40,000 g for ten minutes. The final pellet is resuspended in assay buffer (50 mM Tris HCl buffer (pH 7.4) containing 1 mM EDTA, 5 mM MgCl₂). Incubations were initiated by the addition of tissue to 96-well plates containing test drugs and 0.4-1 nM [3H]diprenorphine in a final volume of 250 ul). Non-specific binding was determined by radioligand binding in the presence of a saturating concentration of naltrexone (10 uM). After one hour incubation period at room temperature, assay samples were rapidly filtered through Whatman GF/B filters and rinsed with ice-cold 50 mM Tris buffer (pH 7.4). Membrane bound [3H]diprenorphine levels were determined by liquid scintillation counting of the filters in BetaScint. The IC₅₀ value (concentration at which 50% inhibition of specific binding occurs) was calculated by linear regression of the concentration-response data. Ki values were calculated according to the Cheng Prusoff equation, Ki=IC₅₀/(1+(L/Kd)), where L is the concentration of the radioligand used in the experiment and the Kd value is the dissociation constant for the radioligand (determined previously by saturation analysis).

Compounds not exemplified but prepared making non-critical changes to conditions provided herein were tested in the kappa assay discussed above and had a Ki value within the range of 0.2 nM and 10,000 nM.

The compounds of the present invention tested in the kappa assay discussed above had the following specific Ki value(s)/ranges with ranges being provided when there is more than one data point:

| Example | Ki (nM) |
|---|---|
| 4a | 0.58-0.66 |
| 4b | 0.83-6.24 |
| 4c | 0.78-1.34 |
| 4d | 0.93-4.72 |
| 4e | 5.03-6.34 |
| 4f | 2.24 |
| 4g | 3.51 |
| 4h | 5.0-5.3 |
| 4i | 6.59-13.0 |
| 4j | 0.9-2.61 |
| 4k | 1.19-2.34 |
| 4l | 2.19-4.78 |
| 4m | 1.41-6.84 |
| 4n | 2.27-2.76 |
| 4o | 2.11-5.08 |
| 4p | 2.68-6.78 |
| 4q | 5.59-14.8 |
| 4r | 3.46-8.28 |
| 4s | 5.27 |
| 4t | 5.76 |
| 4u | 5.84-12.0 |
| 4v | 6.13 |
| 4w | 10.3 |
| 4x | 12.3 |
| 4y | 12.5-24.8 |

General Experimental Procedures

NMR spectra were recorded on a Varian Unity™ 400 (available from Varian Inc., Palo Alto, Calif.) at room temperature at 400 MHz for proton. Chemical shifts are expressed in parts per million (δ) relative to residual solvent as an internal reference. The peak shapes are denoted as follows: s, singlet; d, doublet; t, triplet; q, quartet; m, multiplet; bs, broad singlet; 2s, two singlets. Atmospheric pressure chemical ionization mass spectra (APCI) were obtained on a Fisons™ Platform II Spectrometer (carrier gas: acetonitrile: available from Micromass Ltd, Manchester, UK). Chemical ionization mass spectra (Cl) were obtained on a Hewleft-Packard™ 5989 instrument (ammonia ionization, PBMS: available from Hewlett-Packard Company, Palo Alto, Calif.). Electrospray ionization mass spectra (ES) were obtained on a Waters™ ZMD instrument (carrier gas: acetonitrile: available from Waters Corp., Milford, Mass.). Where the intensity of chlorine or bromine-containing ions are described, the expected intensity ratio was observed (approximately 3:1 for ³⁵Cl/³⁷Cl-containing ions and 1:1 for ⁷⁹Br/⁸¹Br-containing ions) and the intensity of only the lower mass ion is given. In some cases only representative ¹H NMR peaks are given. MS peaks are reported for all examples. Optical rotations were determined on a PerkinElmer™ 241 polarimeter (available from PerkinElmer Inc., Wellesley, Mass.) using the sodium D line (λ=589 nm) at the indicated temperature and are reported as follows [α]_{D}^{temp}, concentration (c=g/100 ml), and solvent.

Column chromatography was performed with either Baker™ silica gel (40 μm; J. T. Baker, Phillipsburg, N.J.) or Silica Gel 50 (EM Sciences™, Gibbstown, N.J.) in glass columns or in Flash 40 Biotage™ columns (ISC, Inc., Shelton, Conn.) under low nitrogen pressure.

Preparation of Intermediates 1(ii):

To a solution of amine of formula HNR¹R² (19.8 mmol) in anhydrous dichloromethane (CH₂Cl₂) (80 mL) was added triethylamine (2.0 g, 19.8 mmol). This was followed by dropwise addition of bromobenzene sulfonyl chloride (4.21 g, 16.5 mmol) and was stirred overnight at room temperature. The reaction was washed with 1N HCl (2×30 mL), the aqueous layer was extracted with CH₂Cl₂ and dried over MgSO₄, that was removed by filtration and the filtrate was concentrated and the intermediate was taken on to next step without further purification. Intermediates made by this process:

| | R¹⁰ | —NR⁶R⁷ | X⁶ | X⁵ |
|---|---|---|---|---|
| 1(ii)(a) | Br | 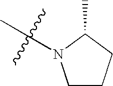 | >C(H)— | >C(H)— |
| 1(ii)(b) | Br | 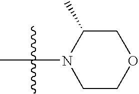 | >C(H)— | >C(H)— |
| 1(ii)(c) | H | 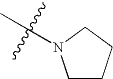 | >N— | >C(H)— |
| 1(ii)(d) | Cl | 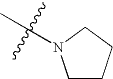 | >C(H)— | >N— |

(R)-1-(2-Bromo-benzenesulfonyl)-2-methyl-pyrrolidine (1(ii)(a)): 84% yield, MS (APCI) (M+1) m/z 305.

(S)-4-(2-Bromo-benzenesulfonyl)-3-methyl-morpholine (1(ii)(b)): 64% yield, MS (APCI) (M+1) m/z 321.

2-(Pyrrolidine-1-sulfonyl)-pyridine (1(ii)(c)): 36% yield, MS (APCI) (M+1) m/z 213.

2-chloro-3-(pyrrolidine-1-yl-sulfonyl)pyridine (1(ii)(d)): 96% yield, MS (APCI) (M+1) m/z 246.

Preparation of Intermediates 2(ii):

3-Bromo-2-(pyrrolidine-1-sulfonyl)-pyridine (2(ii), where —NR⁶R⁷ is pyrrolidinyl): 21% yield, MS (APCI) (M+1) m/z 292:

To a solution of lithium diisopropylamide mono THF in cyclohexane (7.54 mL, 11.3 mmol) in tetrahydrofuran (5 mL) at −60 C was added a solution of the 1 c (1.2 g, 5.7 mmol) in tetrahydrofuran (10 mL) at −60 C and was allowed to stir for 1-1.5 hrs. Bromine (1.8 g, 11.3 mmol) was added dropwise and stirred at −60 C for 1 hr. The reaction was quenched with water at −60 C and warmed to room temperature. The solution was extracted with ethyl acetate, dried and concentrated to give crude oil. Chromatographed on silica using a 0-50% ethyl acetate/heptane gradient.

Preparation of Intermediates 3(iii):

Method III:

To 16.4 ml of a 2M aq sodium carbonate solution was added a solution of 25 mL of dimethylacetamide/dimethyl ethylene glycol, bromo or chloro sulfonamide (2 g, 6.6 mmol) and boronic acid (1.48 g, 9.86 mmol). Finally, [1,1′-Bis(diphenylphosphino)ferrocene]dichloropalladium (II) (53.7 mg, 0.0657 mmol) was added and then heated overnight at 100° C. Reaction was filtered through a fritted funnel and rinsed with water and copious amounts of ethyl acetate. The filtrate was concentrated to ¼ the volume and poured into seperarory funnel. The aqueous phase was extracted with ethyl acetate (3×25 mL) and the organic phase was dried over sodium sulfate, filtered and concentrated. The crude product was chromatographed on silica gel using 10-40% ethyl acetate/heptane gradient.

Method IV:

To 1.95 ml of a 2M aq sodium carbonate solution was added a solution of 2 mL of dimethylacetamide/dimethyl ethylene glycol, bromo sulfonamide (500 mg, 1.56 mmol) and boronic acid (351 mg, 2.34 mmol). Finally, [1,1′-Bis(diphenylphosphino)ferrocene]dichloropalladium (II) (12.8 mg, 0.0156 mmol) was added and then the reaction was microwaved at 100-140 C for 20-40 minutes. Reaction was filtered through a fritted funnel and rinsed with water and copious amounts of ethyl acetate. The filtrate was concentrated to ¼ the volume and poured into seperarory funnel. The aqueous phase was extracted with ethyl acetate (3×25 mL) and the organic phase was dried over sodium sulfate, filtered and concentrated. The crude product was chromatographed on silica gel using 10-40% ethyl acetate/heptane gradient.

| Intermediate | Starting | | | | | |
|---|---|---|---|---|---|---|
| prepared | Material | Method | R⁸ | X⁶ | X⁵ | —NR⁶R⁷ |
| 3(iii)(a) | 1(ii)(a) | III | —H | >C(H)— | >C(H)— | 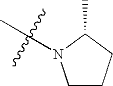 |
| 3(iii)(b) | 1(ii)(b) | IV | —H | >C(H)— | >C(H)— | 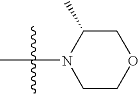 |
| 3(iii)(c) | 2(ii)(a) | III | —H | >N— | >C(H)— | 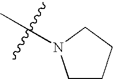 |
| 3(iii)(d) | 1(ii)(b) | IV | —F | >C(H)— | >C(H)— | 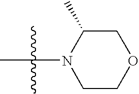 |
| 3(iii)(e) | | IV | —OCH₃ | >C(H)— | >C(H)— | 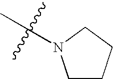 |
| 3(iii)(f) | | III | —H | >C(H)— | >C(H)— | 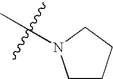 |
| 3(iii)(g) | 1(ii)(d) | III | —H | >C(H)— | >N— | 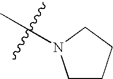 |

2′-((R)-2-Methyl-pyrrolidine-1-sulfonyl)-biphenyl-4-carbaldehyde (3(iii)(a)): <99% yield, MS (APCI) (M+1) m/z 329.

2′-((R)-3-Methyl-morpholine-4-sulfonyl)-biphenyl-4-carbaldehyde (3(iii)(b):) 92.7% yield, MS (APCI) (M+1) m/z 346.

4-[2-(Pyrrolidine-1-sulfonyl)-pyridin-3-yl]-benzaldehyde (3(iii)(c)): 85.4% yield, MS (APCI) (M+1) m/z 317.

3-Fluoro-2′-((R)-3-methyl-morpholine-4-sulfonyl)-biphenyl-4-carbaldehyde (3(iii)(d)): <99% yield, MS (APCI) (M+1) m/z 364.

3-Methoxy-2′-(pyrrolidine-1-sulfonyl)-biphenyl-4-carbaldehyde (3(iii)(e)): 74.0% yield, MS (APCI) (M+1) m/z 346.

2′-(Pyrrolidine-1-sulfonyl)-biphenyl-4-carbaldehyde (3(iii)(f)): 97.5% yield, MS (APCI) (M+1) m/z 316.

4-[3-(pyrrolidin-1-ylsulfonyl)-pyridin-2-yl]benzaldehyde 3(iii)(g): 28% yield, MS (APCI) (M+1) m/z 317.

Preparation of Compounds of Formula I:

Method V:

Aldehyde (3(iii)) (50 mg, 0.159 mmol) was dissolved in 2 mL of anhydrous tetrahydrofuran or dichloromethane followed by the addition of amine (29.5 mg. 0.200 mmol). Catalytic acetic acid (5 uL) and 4 A molecular sieves were added to the solution and allowed to shake for 15-30 minutes. After which time was added MP-triacetoxyborohydride (180 mg, 0.396 mmol). The reaction was shaken overnight at room temperature. MP-carbonate (300 mg, 0.765 mmol) was added to scavenge excess acetic acid along with 5 eq of the respective scavenging agent (PS-isocyanate resin for secondary amines or PS-benzaldehyde for primary amines) and let shake overnight. Reaction was loaded onto a pre-equilibrated (with methanol) Waters Oasis 500 mg MCX cartridge and washed with methanol (10×2 mL). Compounds of formula I were released from the column using 1N ammonia in methanol solution (5×2 mL). Product was isolated and chromatographed on silica gel using 1-10% methanol/dichloromethane gradient.

Method VI:

Aldehyde (3(iii)) (20 mg, 0.063 mmol) was dissolved in 2 mL of dichloromethane followed by addition of amine (9.6 mg, 0.0950 mmol), and the reaction was stirred for about 30 minutes, after which time was added sodium triacetoxyborohydride (26.7 mg, 0.126 mmol) and stirred overnight at room temperature. The reaction was quenched with ethyl acetate (3 mL) and water (3 mL) and allowed to stir for ten minutes after which time the aqueous layer was made basic by adding 10% ammonium hydroxide solution. The solution was extracted with ethyl acetate (3×5 mL). The organic layer was dried over sodium sulfate, filtered and concentrated. Compounds of formula I were chromatographed on silica gel using 5-20% methanol/dichloromethane gradient.

### Examples

4(a): (1R,4S)-Bicyclo[2.2.1]hept-2-yl-[2′-(pyrrolidine-1-sulfonyl)-biphenyl-4-ylmethyl]-amine. Prepared by Method V starting with 3(iii)(f) and bicyclo[2.2.1]heptan-2-amine, giving 4(a) in 63.4% yield. ¹H NMR (400 MHz, CHLOROFORM-d) δ ppm 1.08-1.18 (m, 1 H), 1.22 (br. s., 2 H), 1.25-1.32 (m, 1 H), 1.38-1.49 (m, 2 H), 1.48-1.54 (m, 4 H), 1.59-1.70 (m, 1 H), 1.74-1.86 (m, 1 H), 2.02-2.09 (m, 1 H), 2.19-2.26 (m, 1 H), 2.62-2.74 (m, 4 H), 2.91-3.01 (m, 1 H), 3.63 (t, 2 H), 7.13-7.21 (m, 2 H), 7.32-7.39 (m, 2 H), 7.40-7.49 (m, 2 H), 7.99-8.06 (m, 2 H). MS (APCI) (M+1) m/z 411.

4(b): (1S,5R)-6-[2′-(Pyrrolidine-1-sulfonyl)-biphenyl-4-ylmethyl]-6-aza-bicyclo[3.2.1]octane. Prepared by Method VI starting with 3(iii)(f) and 6-azabicyclo[3.2.1]octane, giving 4(b) in 37.8% yield. ¹H NMR (400 MHz, METHANOL-d₄) δ ppm 1.13-1.22 (m, 1 H), 1.64-1.98 (m, 8 H), 2.27-2.39 (m, 2 H), 2.63-2.77 (m, 2 H), 2.82-2.94 (m, 4 H), 3.36-3.44 (m, 2 H), 3.45-3.54 (m, 1 H), 3.56-3.64 (m, 2 H), 3.95-4.03 (m, 2 H), 4.42 (s, 2 H), 7.31-7.39 (m, 2 H), 7.52 (d, 2 H), 7.56-7.72 (m, 2 H), 8.02-8.10 (m, 2 H). MS (APCI) (M+1) m/z 411.

4(c): 4-Methyl-1-[2′-(pyrrolidine-1-sulfonyl)-biphenyl-4-ylmethyl]-piperidin-4-ol. Prepared by Method V starting with 3(iii)(f) and 4-methylpiperidin-4-ol, giving 4(c) in 75.3% yield. ¹H NMR (400 MHz, CHLOROFORM-d) δ ppm 1.15 (s, 3 H), 1.42-1.55 (m, 6 H), 1.57 (dd, J=10.17, 3.94 Hz, 2 H), 2.24-2.36 (m, 2 H), 2.41-2.53 (m, 2 H), 2.60-2.72 (m, 4 H), 3.47 (s, 2 H), 7.17-7.23 (m, 2 H), 7.34-7.40 (m, 2 H), 7.43-7.50 (m, 2 H), 8.01-8.09 (m, 2 H). MS (APCI) (M+1) m/z 415.

4(d): Isobutyl-[2′-(pyrrolidine-1-sulfonyl)-biphenyl-4-ylmethyl]-amine. Prepared by Method VI starting with 3(iii)(f) and 2-methylpropan-1-amine, giving 4(d) in 82.1% yield. ¹H NMR (400 MHz, CHLOROFORM-d) δ ppm 1.03-1.15 (m, 6 H), 1.59-1.76 (m, 4 H), 2.21-2.35 (m, 1 H), 2.64 (br. s., 2 H), 2.79-2.89 (m, 4 H), 4.24 (br. s., 2 H), 7.22-7.27 (m, 1 H), 7.43-7.60 (m, 4 H), 7.67-7.77 (m, 2 H), 8.08-8.14 (m, 1 H). MS (APCI) (M+1) m/z 373.

4(e): 3,3-Dimethyl-1-[2′-(pyrrolidine-1-sulfonyl)-biphenyl-4-ylmethyl]-piperidine. Prepared by Method V starting with 3(iii)(f) and 3,3-dimethylpiperidine giving 4(e) in 84.0% yield. ¹H NMR (400 MHz, CHLOROFORM-d) δ ppm 0.84 (s, 6 H), 1.08-1.20 (m, 2 H), 1.41-1.58 (m, 6 H), 1.93 (br. s., 2 H), 2.25 (br. s., 2 H), 2.59-2.72 (m, 4 H), 3.38 (s, 2 H), 7.21 (d, J=7.47 Hz, 2 H), 7.37 (d, J=1.66 Hz, 2 H), 7.45 (dd, J=7.47, 1.66 Hz, 2 H), 8.07 (d, J=7.88 Hz, 2H). MS (APCI) (M+1) m/z 413.

4(f): (3-Methyl-oxetan-3-ylmethyl)-[2′-(pyrrolidine-1-sulfonyl)-biphenyl-4-ylmethyl]-amine. Prepared by Method VI starting with 3(iii)(f) and 1-(3-methyloxetan-3-yl)methanamine, giving 4(f) in 38.0% yield. ¹H NMR (400 MHz, CHLOROFORM-d) δ ppm 1.33 (s, 3 H), 1.59-1.68 (m, 4 H), 2.78-2.86 (m, 4 H), 3.84 (s, 2 H), 3.89 (s, 2 H), 4.38 (d, 2 H), 4.48 (d, 2 H), 7.28-7.43 (m, 4 H), 7.45-7.52 (m, 1 H), 7.54-7.61 (m, 1 H), 8.11-8.17 (m, 1 H). MS (APCI) (M+1) m/z 401.

4(g): ((S)-2-Methoxy-1-methyl-ethyl)-[2′-(pyrrolidine-1-sulfonyl)-biphenyl-4-ylmethyl]-amine. Prepared by Method VI starting with 3(iii)(f) and (2S)-1methoxypropan-2-amine, giving 4(g) in 47.0% yield. ¹H NMR (400 MHz, CHLOROFORM-d) δ ppm 1.46 (s, 3 H), 1.66 (s, 4 H), 2.82 (s, 4 H), 3.27 (br. s., 1 H), 3.42 (s, 3 H), 3.60 (br. s., 1 H), 3.85 (br. s., 1 H), 4.11-4.49 (m, 2 H), 7.15-7.24 (m, 1 H), 7.38-7.60 (m, 4 H), 7.75 (br. s., 2 H), 8.10 (d, 1 H). MS (APCI) (M+1) m/z 389.

4(h): [2′-(Pyrrolidine-1-sulfonyl)-biphenyl-4-ylmethyl]-(tetrahydro-pyran-4-yl)-amine. Prepared by Method VI starting with 3(iii)(f) and tetrahydro-2H-pyran-4-amine, giving 4(h) in 87.0% yield. ¹H NMR (400 MHz, METHANOL-d₄) δ ppm 1.63-1.84 (m, 4 H), 2.06-2.19 (m, 2 H), 2.87-2.99 (m, 3 H), 3.24-3.36 (m, 4 H), 3.39-3.54 (m, 2 H), 4.00-4.12 (m, 2 H), 4.31 (s, 2 H), 7.30-7.36 (m, 1 H), 7.46-7.63 (m, 5 H), 7.63-7.71 (m, 1 H), 8.03 (d, 1 H). MS (APCI) (M+1) m/z 400.

4(i): [2′-(Pyrrolidine-1-sulfonyl)-biphenyl-4-ylmethyl]-(tetrahydro-furan-3-yl)-amine. Prepared by Method VI starting with 3(iii)(f) and tetrahydrofuran-3-amine, giving 40) in 27.1% yield. ¹H NMR (400 MHz, CHLOROFORM-d) δ ppm 1.42-1.58 (m, 4 H), 1.63-1.79 (m, 1 H), 1.94-2.09 (m, 1 H), 2.59-2.79 (m, 4 H), 3.25-3.44 (m, 1 H), 3.50-3.62 (m, 1 H), 3.62-3.78 (m, 4 H), 3.79-3.92 (m, 1 H), 7.11-7.21 (m, 2 H), 7.32-7.39 (m, 2 H), 7.41-7.48 (m, 2 H), 7.97-8.04 (m, 2 H). MS (APCI) (M+1) m/z 387.

4(j): Isobutyl-[2′-((R)-2-methyl-pyrrolidine-1-sulfonyl)-biphenyl-4-ylmethyl]-amine. Prepared by Method VI starting with 3(iii)(a) and 2-methylpropan-1-amine, giving 40) in 77.9% yield. ¹H NMR (400 MHz, METHANOL-d₄) δ ppm 0.96 (d, 3 H), 1.05 (d, 6 H), 1.40-1.52 (m, 2 H), 1.60-1.71 (m, 1 H), 1.73-1.92 (m, 2 H), 2.00-2.12 (m, 1 H), 2.85-3.04 (m, 3 H), 3.53-3.61 (m, 1 H), 4.28 (br. s., 2 H), 7.33 (dd, 2 H), 7.45-7.76 (m, 4 H), 8.05 (d, 2 H). MS (APCI) (M+1) m/z 387

4(k): (3,3-Dimethyl-butyl)-[2′-((R)-3-methyl-morpholine-4-sulfonyl)-biphenyl-4-ylmethyl]-amine. Prepared by Method V starting with 3(iii)(b) and 3,3-dimethylbutan-1-amine, giving 4(k) in 36.1% yield. ¹H NMR (400 MHz, CHLOROFORM-d) δ ppm 0.82-0.96 (m, 9 H), 1.06-1.19 (m, 3 H), 1.58-1.71 (m, 2 H), 2.67-2.79 (m, 1 H), 2.85-3.03 (m, 3 H), 3.03-3.16 (m, 2 H), 3.30-3.40 (m, 2 H), 3.50-3.61 (m, 1 H), 4.10 (s, 2 H), 7.19-7.26 (m, 1 H), 7.41-7.52 (m, 4 H), 7.53-7.61 (m, 1 H), 8.10-8.15 (m, 1 H), 8.39 (s, 1 H). MS (APCI) (M+1) m/z 431.

4(l): Isobutyl-[2′-((S)-3-methyl-morpholine-4-sulfonyl)-biphenyl-4-ylmethyl]-amine. Prepared by Method VI starting with 3(iii)(b) and 2-methylpropan-1-amine, giving 4(l) in 42.2% yield. ¹H NMR (400 MHz, METHANOL-d₄) δ ppm 1.03 (d, 6 H), 1.16 (d, 3 H), 1.97-2.15 (m, 1 H), 2.72-2.83 (m, 1 H), 2.87-2.97 (m, 2 H), 2.98-3.10 (m, 1 H), 3.10-3.23 (m, 2 H), 3.23-3.45 (m, 2 H), 3.53-3.65 (m, 1 H), 4.29 (s, 2 H), 7.36 (d, 1 H), 7.51-7.64 (m, 5 H), 7.66-7.76 (m, 1 H), 8.14 (d, 1 H). MS (APCI) (M+1) m/z 403.

4(m): (1S,5R)-6-[3-Fluoro-2′-(pyrrolidine-1-sulfonyl)-biphenyl-4-ylmethyl]-6-aza-bicyclo[3.2.1]octane. Prepared by Method VI starting with 3(iii)(f) and 6-azabicyclo[3.2.1]octane, in 58.1% yield. ¹H NMR (400 MHz, METHANOL-d₄) δ ppm 1.10-1.25 (m, 1H), 1.60-2.01 (m, 8 H), 2.06-2.40 (m, 1 H), 2.62-2.84 (m, 2 H), 2.87-3.05 (m, 4 H), 3.38-3.62 (m, 1 H), 3.63-3.76 (m, 1 H), 3.97 (br. s., 1 H), 4.43-4.68 (m, 3 H), 7.30-7.42 (m, 3 H), 7.58-7.82 (m, 3 H), 8.00-8.08 (m, 1 H). MS (APCI) (M+1) m/z 429.

4(n): 2-{1-[2′-(2-Hydroxymethyl-piperidine-1-sulfonyl)-biphenyl-4-ylmethyl]-piperidin-4-yl}-ethanol. Prepared by Method VI, starting with 2′-{[2-(hydroxymethyl)piperidin-1-yl]sulfonyl}-biphenyl-4-carbaldehyde and 2-piperidin-4-ylethanol, giving 4(n) in 67.0% yield. ¹H NMR (400 MHz, METHANOL-d₄) δ ppm 1.09-1.27 (m, 2 H), 1.35-1.58 (m, 6 H), 1.64-1.88 (m, 2 H), 1.94-2.08 (m, 2 H), 2.64-2.79 (m, 1 H), 2.97-3.12 (m, 3 H), 3.38-3.58 (m, 5 H), 3.56-3.69 (m, 3 H), 4.37 (br. s., 2 H), 7.34 (d, 1 H), 7.54-7.63 (m, 5 H), 7.64-7.71 (m, 1 H), 8.14 (d, 1 H). MS (APCI) (M+1) m/z 473.

4(o): Bicyclo[2.2.1]hept-2-yl-[3-fluoro-2′-((R)-3-methyl-morpholine-4-sulfonyl)-biphenyl-4-ylmethyl]-amine. Prepared by Method V starting with 3(iii)(d) and 3,3-dimethylbutan-1-amine, giving 4(o) in 26.0% yield. ¹H NMR (400 MHz, CHLOROFORM-d) δ ppm 0.91 (s, 9 H), 1.17 (d, 3 H), 1.60-1.71 (m, 2 H), 2.77-2.87 (m, 1 H), 2.89-2.99 (m, 1 H), 3.01-3.11 (m, 1 H), 3.13-3.26 (m, 2 H), 3.38-3.49 (m, 2 H), 3.59-3.69 (m, 1 H), 4.19 (s, 2 H), 7.21-7.33 (m, 2 H), 7.52 (dd, 1 H), 7.60 (dd, 2 H), 8.12 (dd, 1 H), 8.39 (s, 2 H). MS (APCI) (M+1) m/z 449.

4(p): Bicyclo[2.2.1]hept-2-yl-[3-fluoro-2′-((R)-3-methyl-morpholine-4-sulfonyl)-biphenyl-4-ylmethyl]-amine. Prepared by Method V starting with 3(iii)(d) and bicyclo[2.2.1]heptan-2-amine, giving 4(p) in 35.1% yield. ¹H NMR (400 MHz, CHLOROFORM-d) δ ppm 0.66-0.80 (m, 1 H), 1.16 (d, 3 H), 1.19-1.28 (m, 1 H), 1.28-1.51 (m, 2 H), 1.52-1.66 (m, 1 H), 1.72-1.83 (m, 1 H), 1.88-2.01 (m, 2 H), 2.00-2.14 (m, 1 H), 2.17-2.31 (m, 2 H), 2.40-2.48 (m, 1 H), 2.57 (br. s., 1 H), 2.63 (s, 1 H), 2.81 (d, 1 H), 2.99-3.11 (m, 1 H), 3.15-3.26 (m, 2 H), 3.33-3.49 (m, 3 H), 3.60-3.69 (m, 1 H), 4.06-4.22 (m, 3 H), 7.19-7.32 (m, 1 H), 7.48-7.55 (m, 1 H), 7.56-7.65 (m, 2 H), 8.08-8.15 (m, 1 H), 8.34 (s, 2 H). MS (APCI) (M+1) m/z 459.

4(q): [3-Fluoro-2′-((S)-3-methyl-morpholine-4-sulfonyl)-biphenyl-4-ylmethyl]-isobutyl-amine. Prepared by Method VI starting with 3(iii)(d) and 2-methylpropan-1-amine, starting with 3(iii)(d) giving 4(q) in 45.6% yield. ¹H NMR (400 MHz, METHANOL-d₄) δ ppm 1.06 (d, 6 H), 1.18 (d, 3 H), 2.02-2.17 (m, 1 H), 2.81-2.90 (m, 1 H), 2.93-3.01 (m, 2 H), 3.05-3.16 (m, 1 H), 3.17-3.28 (m, 2 H), 3.39-3.50 (m, 2 H), 3.60-3.70 (m, 1 H), 4.37 (s, 2 H), 7.35-7.43 (m, 3 H), 7.59-7.67 (m, 2 H), 7.69-7.76 (m, 1 H), 8.11-8.16 (m, 1 H). MS (APCI) (M+1) m/z 421.

4(r): [3-Fluoro-2′-((R)-2-methyl-pyrrolidine-1-sulfonyl)-biphenyl-4-ylmethyl]-isobutyl-amine. Prepared by Method VI starting with 3(iii)(a) and 2-methylpropan-1-amine, giving 4(r) in 95.0% yield. ¹H NMR (400 MHz, METHANOL-d₄) δ ppm 1.01 (d, 4 H), 1.08 (d, 7 H), 1.46-1.58 (m, 1 H), 1.64-1.77 (m, 1 H), 1.78-2.00 (m, 3 H), 2.00-2.15 (m, 1 H), 2.87-3.14 (m, 5 H), 3.57-3.77 (m, 1 H), 4.35 (s, 2 H), 7.30-7.39 (m, 3 H), 7.57-7.66 (m, 2 H), 7.66-7.74 (m, 1 H), 8.02-8.07 (m, 1 H). MS (APCI) (M+1) m/z 405.

4(s): (3,3-Dimethyl-butyl)-{4-[2-(pyrrolidine-1-sulfonyl)-pyridin-3-yl]-benzyl}-amine. Prepared by Method V starting with 3(iii)(c) and 3,3-dimethylbutan-1-amine, giving 4(s) in 64.0% yield. ¹H NMR (400 MHz, CHLOROFORM-d) δ ppm 0.89 (s, 9 H), 1.45-1.56 (m, 2 H), 1.95-2.04 (m, 4 H), 2.67-2.78 (m, 2 H), 3.45-3.56 (m, 4 H), 3.89 (s, 2 H), 7.40-7.53 (m, 5 H), 7.70-7.77 (m, 1 H), 8.49-8.57 (m, 1 H). MS (APCI) (M+1) m/z 402.

4(t): (5S,6R)-Bicyclo[2.2.1]hept-2-yl-{4-[2-(pyrrolidine-1-sulfonyl)-pyridin-3-yl]-benzyl}-amine. Prepared by Method V starting with 3(iii)(c) and bicyclo[2.2.1]heptan-2-amine, giving 4(t) in 17.0% yield. ¹H NMR (400 MHz, CHLOROFORM-d) δ ppm 0.67-0.77 (m, 1 H), 1.18-1.29 (m, 1 H), 1.29-1.44 (m, 1 H), 1.48-1.61 (m, 1 H), 1.66-1.81 (m, 3 H), 1.88-1.98 (m, 1 H), 1.98-2.04 (m, 4 H), 2.13-2.21 (m, 1 H), 2.30-2.40 (m, 1 H), 3.07-3.17 (m, 1 H), 3.45-3.56 (m, 4 H), 3.76 (dd, 2 H), 7.40-7.51 (m, 5 H), 7.72-7.79 (m, 1 H), 8.49-8.57 (m, 1 H). MS (APCI) (M+1) m/z 412.

4(u): 4′-Cyclopentylaminomethyl-biphenyl-2-sulfonic acid isopropyl-methyl-amide. Prepared by Method VI starting with 4′-formyl-N-isopropyl-N-methylbiphenyl-2-sulfonamide and cyclopentanamine, giving 4(u) in 40.0% yield. ¹H NMR (400 MHz, CHLOROFORM-d) δ ppm 0.88 (d, 6 H), 1.32-1.63 (m, 4 H), 1.66-1.78 (m, 2 H), 1.81-1.94 (m, 2 H), 2.20 (s, 3 H), 3.09-3.20 (m, 1 H), 3.64-3.76 (m, 1 H), 3.82 (s, 2 H), 7.25-7.32 (m, 1 H), 7.32-7.43 (m, 4 H), 7.42-7.50 (m, 1 H), 7.51-7.59 (m, 1 H), 8.10-8.18 (m, 1 H). MS (APCI) (M+1) m/z 387.

4(v): 4′-(Isobutylamino-methyl)-biphenyl-2-sulfonic acid isopropyl-methyl-amide. Prepared by Method VI starting with starting with 3(iii)(g) and 2-methylpropan-1-amine, giving 4(v) in 24.3% yield. ¹H NMR (400 MHz, METHANOL-d₄) δ ppm 0.90-0.98 (m, 6 H), 0.98-1.11 (m, 6 H), 1.93-2.15 (m, 1 H), 2.26-2.41 (m, 3 H), 2.82-2.98 (m, 2 H), 3.62-3.83 (m, 1 H), 4.27 (br. s., 2 H), 7.27-7.36 (m, 1 H), 7.45-7.63 (m, 5 H), 7.63-7.73 (m, 1 H), 8.02-8.11 (m, 1 H). MS (APCI) (M+1) m/z 375.

4(w): (3,3-Dimethyl-butyl)-{4-[3-(pyrrolidine-1-sulfonyl)-pyridin-2-yl]-benzyl}-amine. Prepared by Method V starting with 3(iii)(g) and 3,3-dimethylbutan-1-amine, giving 4(w) in 52.0% yield. ¹H NMR (400 MHz, CHLOROFORM-d) δ ppm 0.91 (s, 9 H), 1.52-1.59 (m, 2 H), 1.60-1.65 (m, 4 H), 2.67-2.74 (m, 2 H), 2.78-2.86 (m, 4 H), 3.96 (s, 2 H), 7.42-7.47 (m, 1 H), 7.49-7.54 (m, 2 H), 7.58-7.63 (m, 2 H), 8.46 (dd, 1 H), 8.80 (dd, 1 H). MS (APCI) (M+1) m/z 402.

4(x): (1S,4R)-Bicyclo[2.2.1]hept-2-yl-{4-[3-(pyrrolidine-1-sulfonyl)-pyridin-2-yl]-benzyl}-amine. Prepared by Method V starting with 4-[3-(pyrrolidin-1-ylsulfonyl)-pyridin-2-yl]benzaldehyde and bicyclo[2.2.1]heptan-2-amine, giving 4(x) in 67.0% yield. ¹H NMR (400 MHz, CHLOROFORM-d) δ ppm 0.63-0.74 (m, 1 H), 1.16-1.26 (m, 1 H), 1.27-1.42 (m, 3 H), 1.46-1.65 (m, 5 H), 1.67-1.78 (m, 1 H), 1.81-1.93 (m, 1 H), 2.10-2.18 (m, 1 H), 2.24-2.33 (m, 1 H), 2.73-2.85 (m, 4 H), 2.98-3.07 (m, 1 H), 3.75 (dd, 2 H), 7.37-7.45 (m, 3 H), 7.55 (d, 2 H), 8.40-8.48 (m, 1 H), 8.74-8.82 (m, 1 H). MS (APCI) (M+1) m/z 412.

4(y): Isobutyl-[3-methoxy-2′-(pyrrolidine-1-sulfonyl)-biphenyl-4-ylmethyl]-amine. Prepared by Method VI starting with 3(iii)(f) and 2-methylpropan-1-amine, giving 4(y) in 72.0% yield. ¹H NMR (400 MHz, METHANOL-d₄) δ ppm 0.96-1.14 (m, 4 H), 1.15-1.47 (m, 3 H), 1.60-1.90 (m, 4 H), 1.97-2.19 (m, 1 H), 2.76-3.05 (m, 5 H), 3.94 (s, 3 H), 4.28 (s, 2 H), 7.04 (d, 1 H), 7.20 (br. s., 1 H), 7.32-7.50 (m, 2 H), 7.54-7.74 (m, 2 H), 8.05 (d, 1 H). MS (APCI) (M+1) m/z 403.

All of the above recited U.S. patents and publications are incorporated herein by reference.

1. A compound of formula I: 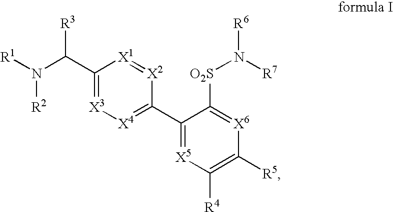
or a pharmaceutically acceptable salt thereof, wherein
R¹ is C₁₋₆alkyl, C₂₋₄alkyl-O—C₁₋₂alkyl, a 7-, 8-, or 9-membered bridged bicyclic carbocyclic ring, a fused bicyclic carbocyclic ring, —(CH₂)ₐ-phenyl, —(CH₂)ₐ-heteroaryl, or —(CH₂)ₐheterocycloalkyl, wherein the bridged ring, the fused ring, phenyl, heteroaryl, or heterocycloalkyl is unsubstituted or substituted with 1, 2, or 3 substituents independently selected from halogen, OH, C₁₋₃alkyl, O—C₁₋₃alkyl, NH₂, NHC₁₋₃alkyl, or N(C₁₋₃alkyl)₂;
R² is H or C₁₋₄alkyl;
or
R¹ and R² are taken together with the nitrogen to which they are attached to form a mono- or bicyclic N-ring, where said N-ring is a 4- to 7-membered mono-cyclic heterocycloalkyl ring; a fused bicyclic heterocyclic ring; or a 7-, 8, or 9-membered bridged bicyclic heterocyclic ring, wherein said N-ring is unsubstituted or substituted with 1, 2, or 3 substituents independently selected from halogen, OH, —CN, C₁₋₃alkyl, C₁₋₃alkyl-OH, O—C₁₋₃alkyl, C₁₋₃alkyl-O—C₁₋₃alkyl, NH₂, NHC₁₋₃alkyl, or N(C₁₋₃alkyl)₂;
a is 0, 1, or 2;
R³ is H, or C₁₋₃alkyl; or
when X¹ or X³ is >(C(R⁸))—, R³ may be taken together with R⁸ of one of X¹ or X³ and the carbon atoms to which they are attached to form a 5- or 6-membered saturated, or partially saturated ring, wherein one carbon atom in said 5- or 6-membered ring may be a heteroatom selected from —O—, —N(H)—, —N(C₁₋₃alkyl)-, and >N—, and wherein said ring is unsubstituted or substituted where valency permits with 1 substituent selected from halogen, —CN, or —C₁₋₃alkyl;
R⁴ is H, halogen, CN, C₁₋₃alkyl, or OC₁₋₃alkyl;
R⁵ is H, halogen, CN, C₁₋₃alkyl, or OC₁₋₃alkyl;
R⁶ is C₁₋₄alkyl, or C₂₋₄alkyl-O—C₁₋₂alkyl;
R⁷ is H, or C₁₋₄alkyl;
or
R⁶ and R⁷ are taken together with the nitrogen to which they are attached to form a mono- or bicyclic SN-ring, where said SN-ring is a 4- to 7-membered mono-cyclic heterocyclic ring containing 0 or 1 additional heteroatom selected from O, NH, or NC₁₋₃alkyl, or a 7-, 8-, or 9-membered bridged bicyclic heterocyclic ring, wherein said SN-ring is unsubstituted or substituted with 1 or 2 substituents independently selected from halogen, OH, —CN, C₁₋₃alkyl, C₁₋₃alkyl-OH, O—C₁₋₃alkyl, C₁₋₃ alkyl-O—C₁₋₃ alkyl, NH₂, NHC₁₋₃alkyl, or N(C₁₋₃alkyl)₂;
X¹, X², X³, X⁴, X⁵, and X⁶ are independently >(C(R⁸)— or >N—; and
each R⁸ is independently H, halogen, —CN, —C₁₋₃alkyl, —OC₁₋₃alkyl, provided that when X¹ or X³ is >(C(R⁸))—, one R⁸ of X₁ or X₃ may be taken together with R³ and the carbon atoms to which they are attached to form said 5- or 6-membered saturated, or partially saturated ring.

2. The compound of claim 1, or pharmaceutically acceptable salt thereof, wherein R⁶ and R⁷ are taken together with the nitrogen to which they are attached to form the monocyclic SN-ring wherein said SN-ring is unsubstituted or substituted with C₁₋₃alkyl.

3. The compound of any of claims 1 to 2, or pharmaceutically acceptable salt thereof, wherein the monocyclic SN-ring is pyrrolidinyl or morpholinyl, and wherein said SN-ring is unsubstituted or substituted with methyl.

4. The compound of claim 3, or pharmaceutically acceptable salt thereof, wherein R¹ is C₁₋₆alkyl or the 7-membered bridged bicyclic carbocyclic ring, and R² is H.

5. The compound of claim 3, or pharmaceutically acceptable salt thereof, wherein R¹ and R² are taken together with the nitrogen to which they are attached to form the 5 to 6-membered mono-heterocycloalkyl ring, or the 8-membered bridged bicyclic heterocylic ring, wherein said N-ring is unsubstituted or substituted with 1 or 2 substitutents independently selected from OH, methyl, or methoxy.

6. The compound of claim 5, or pharmaceutically acceptable salt thereof, wherein X⁶ is N and X¹, X², X³, X⁴, and X⁵ are >(C(R⁸)—, wherein each R⁸ is independently H, halogen, —CN, —C₁₋₃alkyl, or —OC₁₋₃alkyl.

7. The compound of claim 5, or pharmaceutically acceptable salt thereof, wherein X⁵ is N and X¹, X², X³, X⁴, and X⁶ are >(C(R⁸)—, wherein R⁸ is independently H, halogen, —CN, —C₁₋₃alkyl, or —OC₁₋₃alkyl.

8. The compound of claim 5, or pharmaceutically acceptable salt thereof, wherein X¹, X², X³, X⁴, X⁵, and X⁶ are >(C(R⁸))—, where R⁸ is independently H, halogen, —CN, —C₁₋₃alkyl, or —OC₁₋₃alkyl.

9. The compound of claim 8, or pharmaceutically acceptable salt thereof, wherein X¹ or X³ are both >(C(R⁸))— and only one R⁸ of X¹ and X³ is H and the other R⁸ is halogen, —CN, —C₁₋₃alkyl, or —OC₁₋₃alkyl.

10. The compound of claim 1, wherein the compound is selected from the group consisting of
(1R,4S)—N-{[2′-(pyrrolidin-1-ylsulfonyl)biphenyl-4-yl]methyl}bicyclo[2.2.1]heptan-2-amine;
(1R,5S)-6-{[2′-(pyrrolidin-1-ylsulfonyl)biphenyl-4-yl]methyl}-6-azabicyclo[3.2.1]octane;
4-methyl-1-{[2′-(pyrrolidin-1-ylsulfonyl)biphenyl-4-yl]methyl}piperidin-4-ol;
2-methyl-N-{[2′-(pyrrolidin-1-ylsulfonyl)biphenyl-4-yl]methyl}propan-1-amine;
3,3-dimethyl-1-{[2′-(pyrrolidin-1-ylsulfonyl)biphenyl-4-yl]methyl}piperidine;
1-(3-methyloxetan-3-yl)-N-{[2′-(pyrrolidin-1-ylsulfonyl)biphenyl-4-yl]methyl}methanamine;
(2S)-1-methoxy-N-{[2′-(pyrrolidin-1-ylsulfonyl)biphenyl-4-yl]methyl}propan-2-amine;
N-{[2′-(pyrrolidin-1-ylsulfonyl)biphenyl-4-yl]methyl}tetrahydro-2H-pyran-4-amine;
N-{[2′-(pyrrolidin-1-ylsulfonyl)biphenyl-4-yl]methyl}tetrahydrofuran-3-amine;
2-methyl-N-[(2′-{[(2S)-2-methylpyrrolidin-1-yl]sulfonyl}biphenyl-4-yl)methyl]propan-1-amine;
3,3-dimethyl-N-[(2′-{[(3R)-3-methylmorpholin-4-yl]sulfonyl}biphenyl-4-yl)methyl]butan-1-amine;
2-methyl-N-[(2′-{[(3R)-3-methylmorpholin-4-yl]sulfonyl}biphenyl-4-yl)methyl]propan-1-amine;
(1S,5R)-6-{[3-fluoro-2′-(pyrrolidin-1-ylsulfonyl)biphenyl-4-yl]methyl}6-azabicyclo[3.2.1]octane;
2-{1-[(2′-{[2-(hydroxymethyl)piperidin-1-yl]sulfonyl}biphenyl-4-yl)methyl]piperidin-4-yl}ethanol;
N-[(3-fluoro-2′-{[(3R)-3-methylmorpholin-4-yl]sulfonyl}biphenyl-4-yl)methyl]-3,3-dimethylbutan-1-amine;
N-[(3-fluoro-2′-{[(3R)-3-methylmorpholin-4-yl]sulfonyl}biphenyl-4-yl)methyl]bicyclo[2.2.1]heptan-2-amine;
N-[(3-fluoro-2′-{[(3R)-3-methylmorpholin-4-yl]sulfonyl}biphenyl-4-yl)methyl]-2-methylpropan-1-amine;
N-[(3-fluoro-2′-{[(2S)-2-methylpyrrolidin-1-yl]sulfonyl}biphenyl-4-yl)methyl]-2-methylpropan-1-amine;
3,3-dimethyl-N-{4-[2-(pyrrolidin-1-ylsulfonyl)pyridin-3-yl]benzyl}butan-1-amine;
(1R,4S)—N-{4-[2-(pyrrolidin-1-ylsulfonyl)pyridin-3-yl]benzyl}bicyclo[2.2.1]heptan-2-amine;
4′-[(cyclopentylamino)methyl]-N-isopropyl-N-methylbiphenyl-2-sulfonamide;
4′-[(isobutylamino)methyl]-N-isopropyl-N-methylbiphenyl-2-sulfonamide;
3,3-dimethyl-N-{4-[3-(pyrrolidin-1-ylsulfonyl)pyridin-2-yl]benzyl}butan-1-amine;
(1S,4R)—N-{4-[3-(pyrrolidin-1-ylsulfonyl)pyridin-2-yl]benzyl}bicyclo[2.2.1]heptan-2-amine; and
N-{[3-methoxy-2′-(pyrrolidin-1-ylsulfonyl)biphenyl-4-yl]methyl}-2-methylpropan-1-amine;
or a pharmaceutically acceptable salt thereof.

11. A pharmaceutical composition comprising a compound of any of claims 1, 2 and 10, or pharmaceutically acceptable salt thereof, for treating or preventing a disease or disorder selected from the group consisting of schizophrenia including negative symptoms; schizophreniform disorder; schizoaffective disorder including of the delusional type or the depressive type; delusional disorder; substance-induced psychotic disorder; personality disorder of the paranoid type; personality disorder of the schizoid type; panic disorder; phobias; obsessive-compulsive disorder; stress disorders; generalized anxiety disorder; movement disorders involving Huntington's disease; dyskinesia associated with dopamine agonist therapy; Parkinson's disease: restless leg syndrome; disorders comprising as a symptom thereof a deficiency in cognition; dementias; mood disorders and episodes in a mammal; anxiety or psychotic disorders including schizophrenia, of the paranoid, disorganized, catatonic, undifferentiated, or residual type; delusional disorder; personality disorder of the paranoid type, of the schizoid type, or agoraphobia; post-traumatic stress disorder; acute stress disorder; chemical dependencies including alcohol, amphetamine, cocaine, heroin, phenobarbital, opiate, nicotine and benzodiazepines addiction; deficiency in memory, intellect, or learning and logic ability; reduction in any particular individual's functioning in one or more cognitive aspects; age-related cognitive decline; dementia; Alzheimer's disease; multi-infarct dementia; alcoholic dementia or other drug-related dementia; dementia associated with intracranial tumors or cerebral trauma; dementia associated with Huntington's disease or Parkinson's disease; AIDS-related dementia; delirium; amnestic disorder; mental retardation; a learning disorder including reading disorder, mathematics disorder, or a disorder of written expression; attention-deficit/hyperactivity disorder; mood disorders or mood episodes; a manic or mixed mood episode; a hypomanic mood episode; a depressive episode with atypical features; a depressive episode with melancholic features; a depressive episode with catatonic features; a mood episode with postpartum onset; post-stroke depression; dysthymic disorder; minor depressive disorder; premenstrual dysphoric disorder; post-psychotic depressive disorder of schizophrenia; a major depressive disorder superimposed on a psychotic disorder; delusional disorder or schizophrenia; a bipolar disorder including bipolar I disorder, bipolar II disorder, cyclothymic disorder, hypertension, and depression; depression in cancer patients, Parkinson's patients, infertile women, and pediatrics; depression as a single episode depression or recurrent episodes; including depression associated with postmyocardial infarction, subsyndromal symptomatic depression, induced by child abuse, post partum depression, and major depression of the mild, moderate or severe type; avoidant personality disorder; premature ejaculation; eating disorders including anorexia nervosa and bulimia nervosa; obesity; duster headache; migraine; pain; neuroleptic-induced parkinsonism and tardive dyskinesias; endocrine disorders; hyperprolactinaemia; vasospasm; vasospasm in the cerebral vasculature; cerebellar ataxia; gastrointestinal tract disorders involving changes in motility and secretion; mania; premenstrual syndrome; fibromyalgia syndrome; stress incontinence; Tourette's syndrome; trichotillomania; kleptomania; male impotence; cancer; small cell lung carcinoma; chronic paroxysmal hemicrania; and headache associated with vascular disorders and a pharmaceutical carrier.

12. A method of treating or preventing a disease or disorder selected from the group consisting of schizophrenia including negative symptoms; schizophreniform disorder; schizoaffective disorder including of the delusional type or the depressive type; delusional disorder; substance-induced psychotic disorder; personality disorder of the paranoid type; personality disorder of the schizoid type; panic disorder; phobias; obsessive-compulsive disorder; stress disorders; generalized anxiety disorder; movement disorders involving Huntington's disease; dyskinesia associated with dopamine agonist therapy; Parkinson's disease; restless leg syndrome; disorders comprising as a symptom thereof a deficiency in cognition; dementias; mood disorders and episodes in a mammal; anxiety or psychotic disorders including schizophrenia, of the paranoid, disorganized, catatonic, undifferentiated, or residual type; delusional disorder; personality disorder of the paranoid type, of the schizoid type, or agoraphobia; post-traumatic stress disorder; acute stress disorder; chemical dependencies including alcohol, amphetamine, cocaine, heroin, phenobarbital, opiate, nicotine and benzodiazepines addiction; deficiency in memory, intellect, or learning and logic ability; reduction in any particular individual's functioning in one or more cognitive aspects; age-related cognitive decline; dementia; Alzheimer's disease; multi-infarct dementia; alcoholic dementia or other drug-related dementia; dementia associated with intracranial tumors or cerebral trauma; dementia associated with Huntington's disease or Parkinson's disease; AIDS-related dementia; delirium, amnestic disorder; mental retardation; a learning disorder including reading disorder, mathematics disorder, or a disorder of written expression; attention-deficit/hyperactivity disorder; mood disorders or mood episodes; a manic or mixed mood episode; a hypomanic mood episode; a depressive episode with atypical features; a depressive episode with melancholic features; a depressive episode with catatonic features; a mood episode with postpartum onset; post-stroke depression; dysthymic disorder; minor depressive disorder; premenstrual dysphoric disorder; post-psychotic depressive disorder of schizophrenia; a major depressive disorder superimposed on a psychotic disorder; delusional disorder or schizophrenia; a bipolar disorder including bipolar I disorder, bipolar II disorder, cyclothymic disorder, hypertension, and depression; depression in cancer patients, Parkinson's patients, infertile women, and pediatrics; depression as a single episode depression or recurrent episodes; including depression associated with postmyocardial infarction, subsyndromal symptomatic depression, induced by child abuse, post partum depression, and major depression of the mild, moderate or severe type; avoidant personality disorder; premature ejaculation; eating disorders including anorexia nervosa and bulimia nervosa; obesity; cluster headache; migraine; pain; neuroleptic-induced parkinsonism and tardive dyskinesias; endocrine disorders; hyperprolactinaemia; vasospasm; vasospasm in the cerebral vasculature; cerebellar ataxia; gastrointestinal tract disorders involving changes in motility and secretion; mania; premenstrual syndrome; fibromyalgia syndrome; stress incontinence; Tourette's syndrome; trichotillomania; kleptomania; male impotence; cancer; small cell lung carcinoma; chronic paroxysmal hemicrania; and headache associated with vascular disorders by administering to a mammal in need thereof an effective amount of a compound of any of claims 1, 2 and 10, or pharmaceutically acceptable salt thereof.

13. The method of claim 12, wherein the compound of any of claims 1, 2 and 10 is administered with at least one additional pharmaceutical agent.

14. A pharmaceutical composition comprising (i) a therapeutically effective amount of a compound or a pharmaceutically acceptable salt thereof of any of claims 1, 2 and 10; and (ii) at least one a pharmaceutically acceptable excipient, diluent, or carrier.

15. The composition of claim 14 further comprising at least one additional pharmaceutical agent.

16. A method for treating a disease, condition or disorder that is mediated by antagonizing the kappa opioid receptor in animals comprising the step of administering to an animal in need of such treatment a therapeutically effective amount of a compound of any of claims 1, 2 and 10.

17. The method of claim 16 wherein said disease, condition and/or disorder is schizophrenia, depression, or bipolar.

18. The method of claim 16 or 17, wherein the compound of any of claims 1 to 10 is administered with at least one additional pharmaceutical agent.
